(19) United States
(12) Patent Application Publication
(10) Pub. No.: US 2010/0152747 A1
(43) Pub. Date: Jun. 17, 2010

(54) INSERTION SYSTEM AND LEAD FOR TREATMENT OF A TARGET TISSUE REGION

(75) Inventors: Alexander Padiy, Geldrop (NL); Michel Marcel Jose Decre, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., Eindhoven (NL)

(21) Appl. No.: 12/600,489

(22) PCT Filed: Jun. 3, 2008
(86) PCT No.: PCT/IB2008/052163

§ 371 Date: Nov. 17, 2009

Related U.S. Application Data
(60) Provisional application No. 60/941,740, filed on Jun. 4, 2007.

(51) Int. Cl. A61B 17/00

(52) U.S. Cl. 606/129

(57) ABSTRACT

The present disclosure provides for systems and methods enabling the insertion of leads (as used e.g., in a framework of the brain treatment therapies) through a target anatomy for conforming with a target tissue region. An exemplary lead includes at least a partially curved portion for conforming with a geometry defined by the target tissue region. In an exemplary embodiment, the system relates to stimulating targets in the brain for improved post-operative steering of an applied electric field. The leads can be either pre-curved or put under transversal mechanical strain during insertion such that a certain curved curvature of the insertion trajectory is achieved. The system includes at least a first insertion tool removably engaged with respect to the lead for guiding and providing mechanical support to the lead during insertion.

The present disclosure relates to systems and methods for enabling the placement of leads for conforming with geometries defined by target tissue regions of an anatomy and treatment of the target tissue region thereof.

Implantable brain neuro-stimulation devices are increasingly being utilized in clinical practice for several treatments including Parkinson's disease, movement disorders, and epilepsy. Moreover, current research includes testing for neuro-stimulation for treatment of mood and anxiety disorders. These devices use electrical stimulation for exciting or inhibiting certain regions in the brain related to manifestation of the particular disease. Medical practitioners can also envisage the use of chemical or optical stimulation of the brain structures for achieving comparable therapeutic effects.

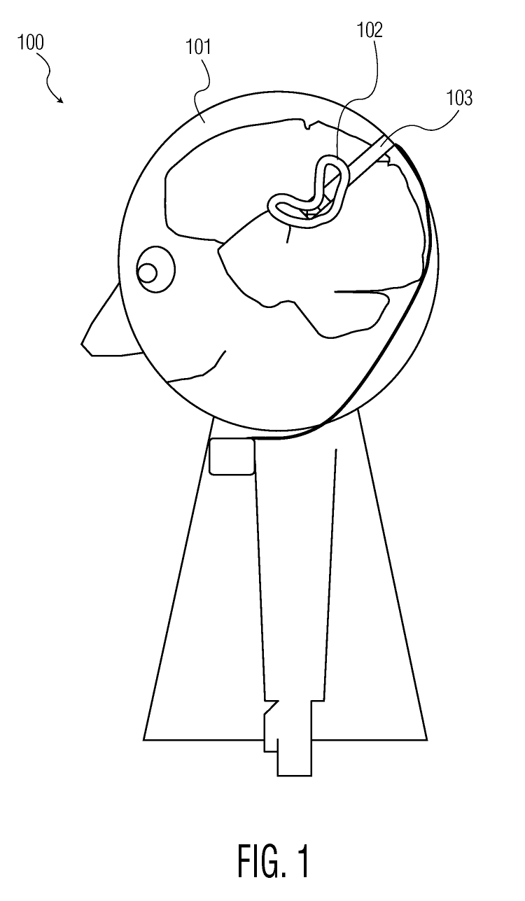

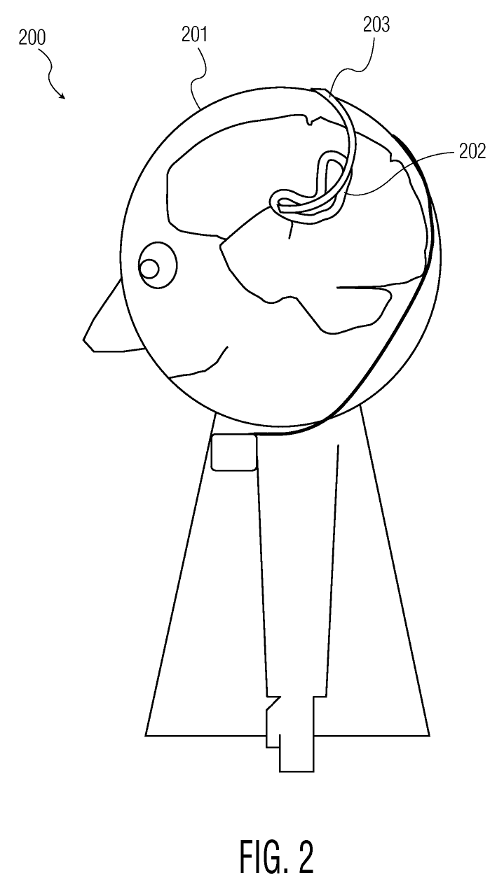

Current practice often use leads, generally made from flexible cables having a diameter of 1 to 2 mm. Moreover, the leads are often equipped with a number of electrical contacts through which the electric currents are being supplied to the brain tissue, as is schematically depicted in FIG. 1. Such leads are positioned in the patient's brain using straight guide-tubes and a mechanical positioning system engaged with respect to a stereotactic frame. As a consequence, leads are typically implanted along straight lines with respect to the burr-hole.

Unfortunately, the straight shape of the stimulation lead after placement and or insertion often does not match well and/or effectively conform with the shape of the target brain regions intended to be stimulated for achieving a desired therapeutic effect. These regions typically define somewhat of a curved shape, e.g. hippocampus that is U-shaped, as shown in FIG. 1. Moreover, steering of the applied electric field is very limited as only lateral field gradients can be created, thus heavily restricting the ability to select the volume of the neural tissue to be stimulated.

In U.S. Pat. No. 6,343,226, a technique is described whereby electrical stimulation to treat symptoms from central and peripheral nervous system disorders such as those found in e.g. Parkinson's disease, epilepsy, psychiatric illness and intractable pain, using a quadric-polar deep brain stimulation electrode connected to an implantable pulse generator have been expanded. By an implantation of an electrode, it is important for the outcome to determine the optimal placement of the electrode. An electrode device is provided allowing stimulation of a large volume of neural tissue in combination with a simultaneous microelectrode recording. Other features involve a temporary electro-physiological micro-recording microelectrode/stilette 1, a bent electrode tip, a split electrode tip or an asymmetrical electrical stimulation field. This technique allows for a less traumatic localization of the optimal neural stimulation area by microelectrode recording in combination with the placement of the permanent deep brain stimulation electrode.

In U.S. Pat. No. 7,033,326, systems and methods of implanting a lead for brain stimulation are described. Leads and introduction tools are proposed for deep brain stimulation and other applications. Some embodiments provide lead designs which may be placed with a stylet, while others do not require a stylet. Some lead embodiments use standard wire conductors, while others use cable conductors. Several embodiments incorporate microelectrodes and/or microelectrode assemblies. Certain embodiments provide introduction tools, such as cannula and/or cannula systems, which ensure proper placement of, e.g., leads.

U.S. Patent Application 2005/0137647 describes a method of intravascularly delivering stimulation leads into direct contact with tissue. According to this application, a method of treating a disorder in a patient includes delivering a stimulation lead within a blood vessel, intralumenally puncturing a wall of the blood vessel to create an exit point, and then introducing the stimulation lead through the exit point into direct contact with tissue the stimulation of which treats the disorder. Optionally, the method includes implanting a source of stimulation within the patient's body, and then electrically coupling the proximal end of the stimulation lead to the implanted stimulation source. Using the stimulation lead, the tissue can then be stimulated in order to treat the disorder.

U.S. Patent Application 2006/0122677 describes various apparatus and methods for deep brain stimulating electrodes. This application describes an apparatus having a deploying deep brain stimulating probe with a shaft, at least one opening on the shaft, at least one extendable tendril deploying from the shaft into surrounding tissue through the opening and an electrode disposed on the tendril.

U.S. Patent Application 2006/0149335 describes devices and methods for brain stimulation. This application describes a device for brain stimulation that includes a lead having a longitudinal surface, at least one stimulation electrode disposed along the longitudinal surface of the lead, and at least one recording electrode, separate from the at least one stimulation electrode, disposed along the longitudinal surface of the lead.

U.S. Patent Application 2004/0186544 describes electrical tissue stimulation apparatus and methods. This application describes an implantable lead for electrical stimulation of tissue having wire-like extendable members whose tips curl back upon themselves in open tissue spaces to form 2- or 3-dimensional electrodes. The electrodes may be positioned axially or in other directions from the lead body. Traction on the lead body or extendable members allows easy withdrawal as the member tip electrodes uncurl, allowing removal without major surgery.

Despite efforts to date, a need still exists for effective insertion/lead combination systems and methods capable of effectively reaching, engaging with and treating target locations. These and other needs are addressed and/or overcome by the systems and methods of the present disclosure.

The present disclosure provides for systems and methods for treatment of target tissue regions associated with a target anatomy. In an exemplary embodiment, a target tissue insertion system includes: (a) a lead adapted to access a target tissue region associated with a target anatomy; and (b) at least a first insertion tool removably engaged with the lead. The target tissue region defines a geometry and the lead defines a curved portion adapted to conform with the geometry of the target tissue region. The insertion tool is adapted to insert the lead into the target anatomy to engage with the target tissue region The insertion tool is removable once the lead is positioned with respect to the target tissue region.

The insertion tool is adapted to provide guidance and mechanical support to the lead during insertion. In an exemplary embodiment, the lead accesses the target tissue region to perform a function selected from the group consisting of stimulating the target tissue region, recording activity associated with the target tissue region and delivering a drug and/or chemical to the target tissue region. The lead can be pre-curved to conform with the geometry of the target tissue region and fabricated so as to be substantially inflexible. The lead can alternatively be fabricated so as to be substantially soft and flexible and adapted to curve so as to conform with the geometry of the target tissue region after being inserted into the target anatomy. In an exemplary embodiment, the target tissue region is at least a portion of a brain enclosed within a skull of a patient.

The present disclosure provides for an exemplary insertion tool that is externally positioned with respect to the lead substantially surrounding the lead. The insertion tool can be adapted to guide the lead to the target tissue region and not penetrate the skull, or guide the lead to the target tissue region and penetrate the skull. In an exemplary embodiment, a cross section of the insertion tool defines a first geometry and a cross section of the lead surrounded by the insertion tool defines a second geometry and the first and second geometries define a geometric relationship. The relationship between the first and second geometries can be similar or non-rotatably symmetric. In an exemplary embodiment, the first geometry is circular and the second geometry is selected from the group consisting of square, elliptical and triangular.

The present disclosure provides for an exemplary lead that is curved defining a geometry selected from the group consisting of an arc of a circle geometry and a corkscrew/helix geometry. The lead defining these geometries typically defines a lead tip that moves along a path through the target anatomy during insertion such that all parts of the lead follow the same path as the lead tip. In an exemplary embodiment, the lead is substantially tube shaped having an opening at a distal end and a closed portion at a proximal end and the at least first insertion tool is positioned internally with respect to the lead. The at least first insertion tool can be any insertion tool capable of conforming the lead with respect to the target tissue region such as a guide wire.

In an exemplary embodiment, the first insertion tool is positioned internal with respect to the lead and the system further includes a second insertion tool positioned internally with respect to the lead surrounding the first insertion tool. In an exemplary embodiment, the first insertion tool can be a guide wire and the second insertion tool can be a syringe or a cannula. A cross section of the first insertion tool defines a first geometry and a cross section of the second insertion tool surrounding the first insertion tool defines a second geometry. The first and second geometries can be similar or define a non-circularly symmetric relationship. In an exemplary embodiment, the second geometry is circular and the first geometry is selected from the group consisting of square, elliptical and triangular.

The present disclosure provides for an exemplary system such that the lead and the at least first insertion tool define similar curved curvatures. In an exemplary embodiment, the lead is pre-curved and includes a straight portion and a curved portion such that the curved portion is at a proximal end with respect to the target tissue region and the straight portion is at a distal end with respect to the target tissue region. In a further exemplary embodiment, the at least first insertion tool is substantially straight and positioned external with respect to the lead. The curved portion remains internal with respect to the insertion tool causing the curved portion to be straightened temporarily until it is further inserted to reach the target tissue region thereby following a substantially curved trajectory path. In an exemplary embodiment, the at least first insertion tool is a guide wire positioned internal with respect to the lead, the at least first insertion tool includes a substantially straight portion at a distal end with respect to the target tissue region and a curved portion at a proximal end with respect to the target tissue region.

The present disclosure provides for an exemplary system having a lead that is fabricated so as to be substantially soft and flexible and includes a straight portion and a curved portion such that the curved portion is at a proximal end with respect to the target tissue region and the straight portion is at a distal end with respect to the target tissue region. The system can include a second insertion tool surrounding the first insertion tool removably positioned internal with respect to the lead. The second insertion tool can be fabricated so as to be substantially inflexible defining a substantially straight trajectory. With respect to a substantially inflexible second insertion tool, the first insertion tool and the lead are straightened by the inflexible second insertion tool during insertion and then move along a substantially curved trajectory path as the second insertion tool is being removed. In an exemplary embodiment, a system according to the present disclosure can include a positioning support apparatus for providing support to the insertion of the insertion tool and lead for reaching the target tissue region.

In an exemplary embodiment, the at least first insertion tool is a guide wire and defines a substantially helical or cork-screw geometry. In a further exemplary embodiment, the lead defines a substantially helical or cork-screw geometry. The at least first insertion tool can be a guide wire positioned internal with respect to the lead and the at least first insertion tool can include a substantially straight portion at a distal end with respect to the target tissue region and a helical or corkscrew portion at a proximal end with respect to the target tissue region. The lead can include a substantially straight portion at a distal end with respect to the target tissue region and a helical or corkscrew portion at a proximal end with respect to the target tissue region.

In an exemplary embodiment, the lead can further include a plurality of wires running through the lead in a substantially longitudinal direction for inducing transversal mechanical strain at least in a distal end of the lead during insertion. The at least first insertion tool further includes a plurality of wires running through the insertion tool in a substantially longitudinal direction for inducing transversal mechanical strain at least in a distal end of the first insertion tool during insertion.

The present disclosure provides for an exemplary method for insertion of a lead into a target tissue region to conform with the target tissue region geometry including the steps of: (a) providing a pre-curved lead or inducing a curved trajectory on a non pre-curved lead; (b) removably engaging at least a first insertion tool with respect to the lead; and (c) inserting the lead and the engaged insertion tool through a target anatomy to reach the target tissue region. The lead is curved so as to conform with the geometry of the target tissue region. The at least first insertion tool can be positioned internal or external with respect to the lead. The lead is adapted to perform a function selected from the group consisting of stimulating the target tissue region, recording activity associated with the target tissue region, and delivering a drug and/or chemical to the target tissue region. The at least a first insertion tool guides the lead to reach the target tissue region.

Additional features, functions and benefits of the disclosed systems and methods will be apparent from the description which follows, particularly when read in conjunction with the appended figures.

To assist those of ordinary skill in the art in making and using the disclosed systems and methods, reference is made to the appended figures, wherein:

FIG. 1 is a schematic illustrating an exemplary traditional implantable medical device associated with prior art applications and systems for deep brain treatment such as stimulation;

FIG. 2 is a schematic illustrating an exemplary insertion system having a curved lead;

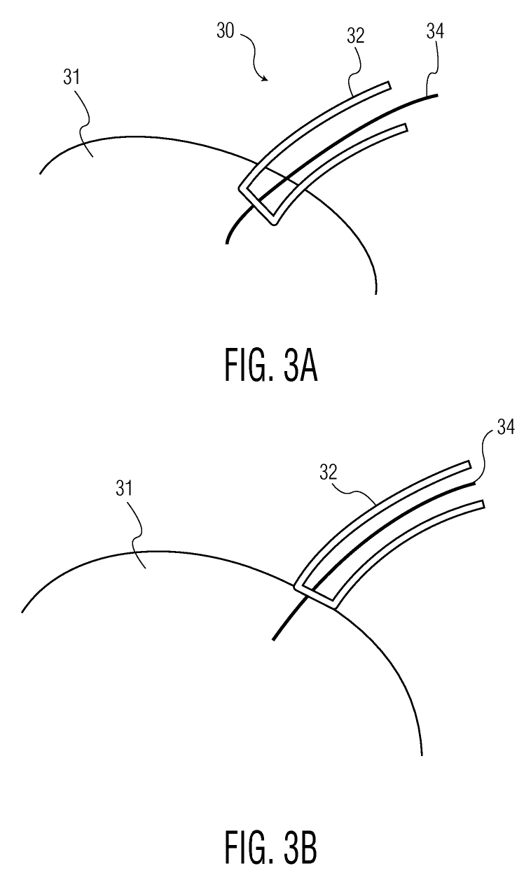

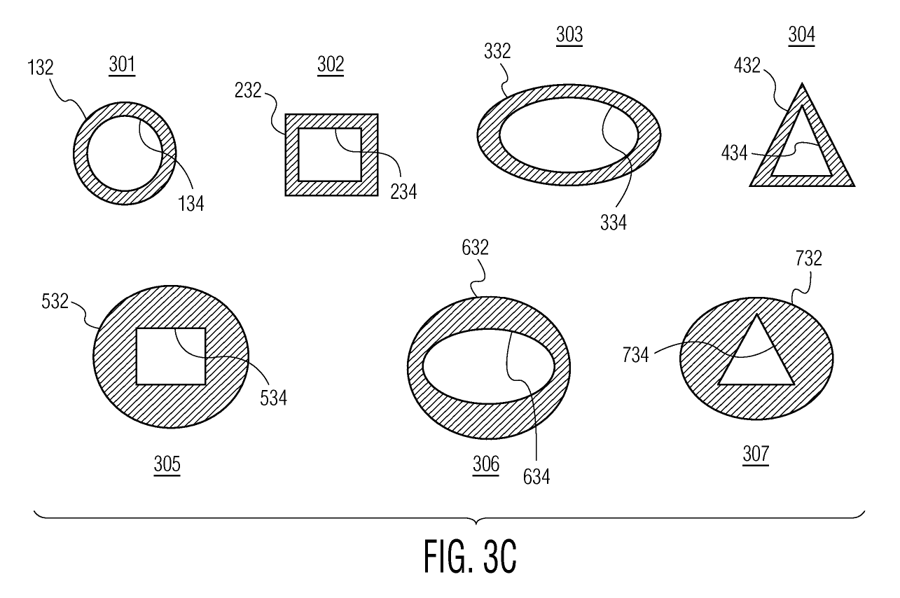

FIG. 3(a) is a schematic illustrating an exemplary insertion system associated with the present disclosure including an external insertion tool in cooperation with a curved lead wherein the insertion tool penetrates the skull;

FIG. 3(b) is a schematic illustrating an exemplary insertion system associated with the present disclosure including an external insertion tool in cooperation with a curved lead wherein the insertion tool does not penetrate the skull;

FIG. 3(c) illustrates exemplary cross section views illustrating the geometric relationships between different exemplary leads surrounded by exemplary insertion tools;

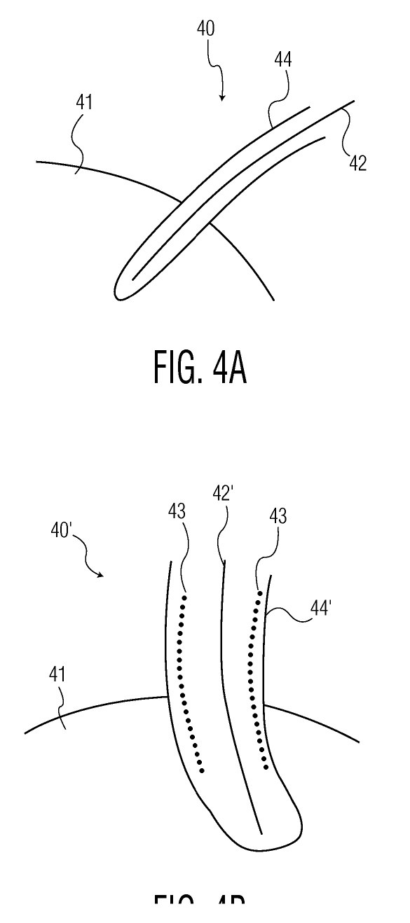

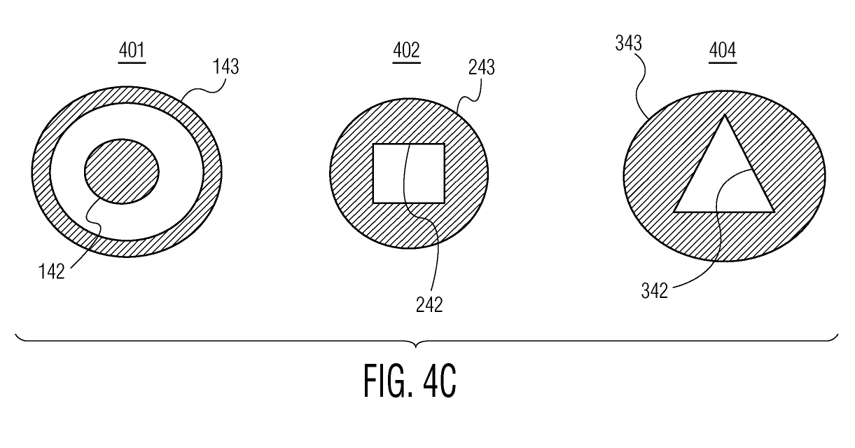

FIG. 4(a) is a schematic illustrating an exemplary insertion system associated with the present disclosure including an internal insertion tool for implanting a substantially soft lead;

FIG. 4(b) is a schematic illustrating an exemplary stimulation system associated with the present disclosure including a first internal insertion tool for implanting a substantially soft lead and a second internal insertion tool for providing additional mechanical support;

FIG. 4(c) illustrates exemplary cross section views illustrating the geometric relationships between different exemplary first and second insertion tools;

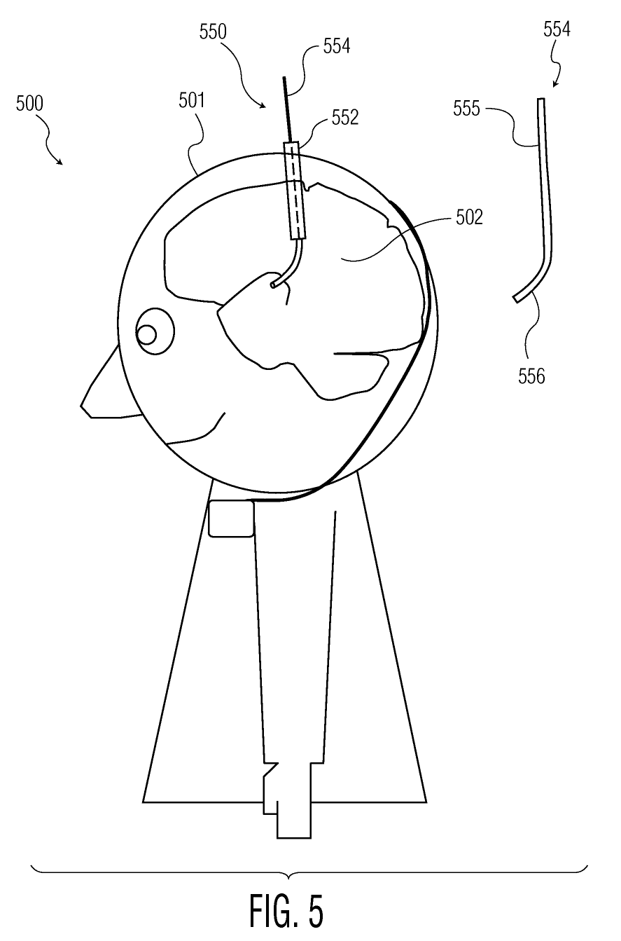

FIG. 5 is a schematic illustrating an exemplary system associated with the present disclosure including a pre-curved lead (or pre-curved guide wire) having an curved portion and a straight portion engaged with respect to an external insertion tool;

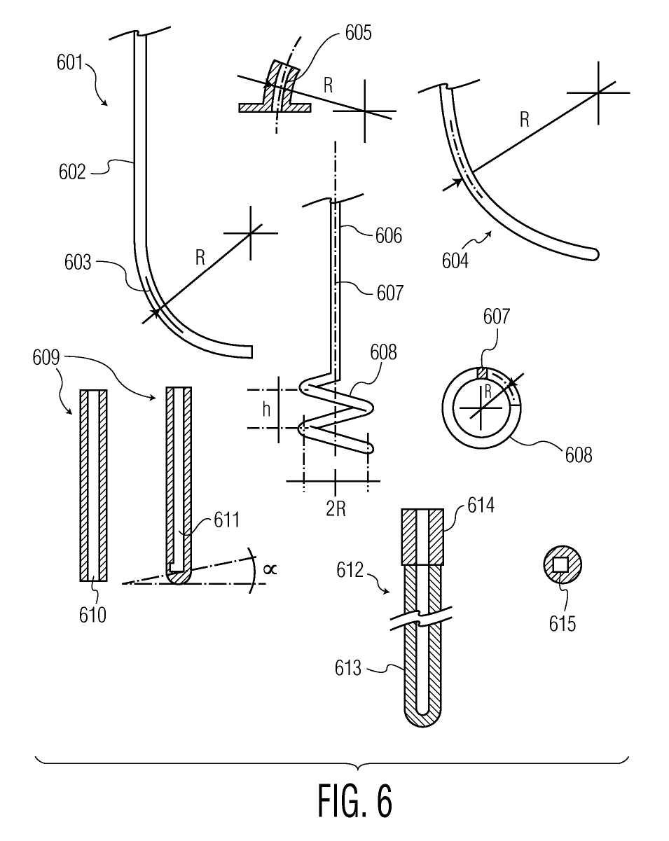

FIG. 6 illustrates several detailed exemplary embodiments of non-preshaped leads and pre shaped insertion tools associated with the present disclosure;

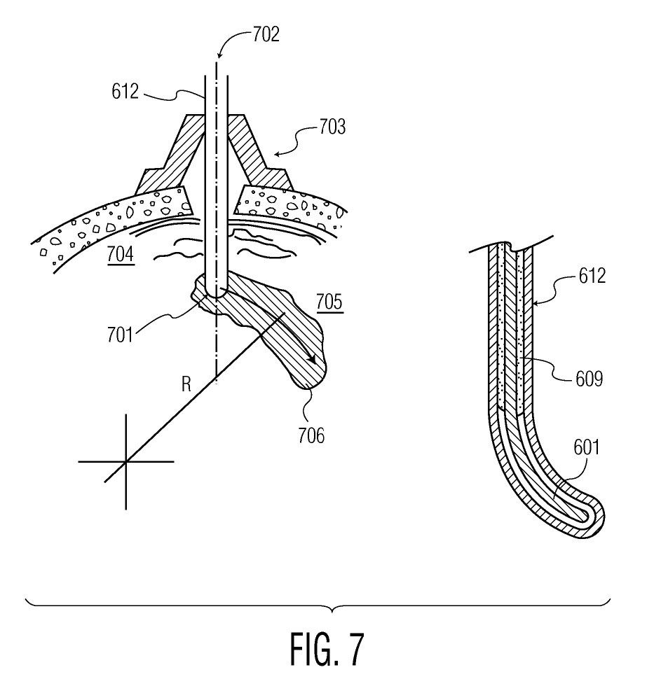

FIG. 7 illustrates a schematic of an exemplary insertion procedure and method for non-preshaped leads associated with the present disclosure;

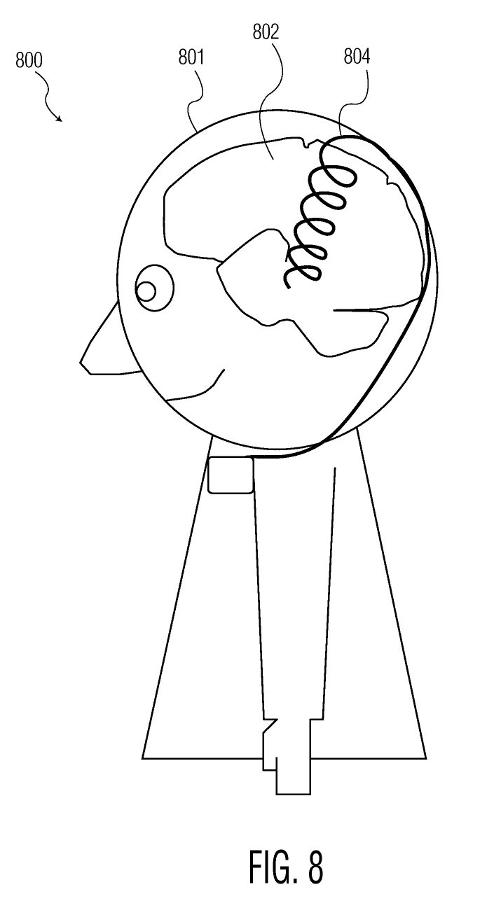

FIG. 8 illustrates a schematic of an exemplary curved lead (or curved guide wire) defining a helical (cork-screw) geometry;

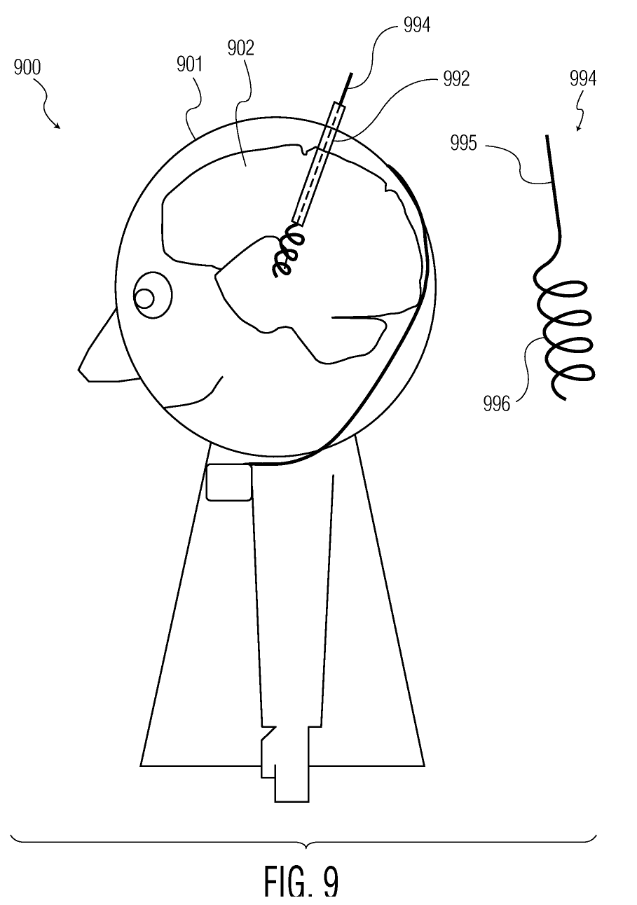

FIG. 9 illustrates a schematic of an exemplary curved lead (or curved guide wire) having a spiral (cork-screw) portion and a straight portion;

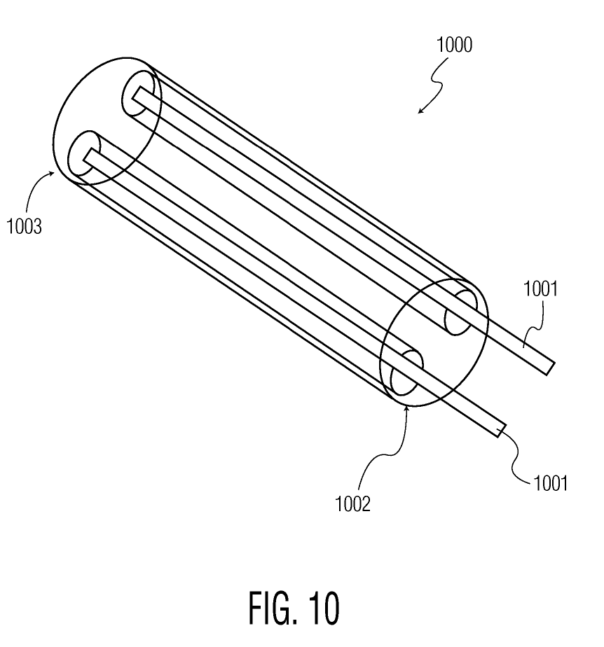

FIG. 10 is a schematic illustrating a system associated with the present disclosure including a substantially flexible lead and a number of wires running through the lead in a longitudinal direction from a distal end to a proximal end for inducing transversal mechanical strain.

The present disclosure provides for systems and methods that utilize at least partially curved leads for conforming with a target tissue region associated with an anatomical region such as a brain. The target tissue region is intended to undergo treatment such as neural stimulation, brain activity recording or drug/chemical delivery as illustrated in FIG. 2 and FIG. 5. In an exemplary embodiment, the leads can be either pre-curved or shaped under transversal mechanical strain during insertion such that a certain curvature of the insertion trajectory is achieved.

FIG. 1 illustrates a traditional substantially straight implantable medical device 103 penetrating a skull 101 of an exemplary patient 100. Deep brain stimulation unit 103 is intended to reach and/or stimulate target tissue region 102. However, since unit 103 is substantially linear (i.e., having no curvature), only a portion of target tissue region 102 is reachable by unit 103.

Although reference is being made to stimulation of a target tissue region of a brain, it is understood that a medical device can be inserted for several other treatments including but not limited to recording of target tissue activity (e.g., brain activity) and drug/chemical delivery. FIG. 2 illustrates an exemplary embodiment associated with the present disclosure showing particular advantages over the prior art systems as illustrated in FIG. 1. A particular advantage associated with the system shown in FIG. 2 includes but is not limited to the lead associated with the therapy insertion unit is able to more effectively conform with an intended geometry defined by the target tissue region. FIG. 2 illustrates a substantially curved brain stimulation unit 203 inserted into a skull 201 associated with an exemplary patient 200. The curved unit 203 is adapted to conform with an exemplary target tissue region 202.

In an exemplary embodiment, a lead having relatively little intrinsic mechanical strain can be used in combination with an insertion tool that facilitates placing the lead along a curved trajectory as illustrated in FIGS. 4, 6, and 7. Utilizing an insertion tool enables the placement and/or insertion of a substantially soft and flexible lead having relatively similar mechanical properties as the properties of soft brain tissue. A lead having similar mechanical properties to a target tissue region can be effective in reducing undesired brain tissue reaction such as scar tissue development or other biocompatibility reactions, which are typical under chronic implantation conditions. In an exemplary embodiment, a combination system includes using a detachable (i.e., removable) pre-strained guide wire in combination with a relatively soft flexible lead (both delivered to the desired location via a delivery unit such as a syringe).

In an exemplary embodiment insertion of a lead into a target location is guided by an insertion tool able to induce transversal mechanical strain in a proximal portion during insertion. The following examples describe particular exemplary embodiments associated with the present disclosure and are not intended to limit the scope of the present disclosure to such embodiments thereof. Rather, as will be readily apparent to persons skilled in the art from the description provided herein, to include modifications, alterations and enhancements without departing from the spirit or scope of the present disclosure.

### EXAMPLE 1

In an exemplary embodiment, a stiff pre-curved lead is inserted into a patient's skull to reach and conform with a target location such as a target brain tissue region. The pre-curved lead is sized and shaped to define at least a partial arc of circle-shape. A partially curved insertion tool (e.g., a syringe) advantageously engages with the lead during implantation to enhance mechanical strength of the overall system and thus improve insertion accuracy.

FIG. 3(a) illustrates an exemplary insertion system 30 associated with the present disclosure. An exemplary system 30 includes an external insertion tool 32 supporting and engaged with an internal lead 34. Insertion tool 32 is adapted to at least partially penetrate an exemplary target region such as a skull. This allows for precise and effective positioning of lead 34 with respect to a target tissue region such as a portion of a brain surrounded by skull 31. The insertion tool 32 illustrated in FIGS. 3(a) and 3(b) is external to lead 34 thus surrounding or at least positioned with respect to the outer surface of lead 34. FIG. 3(b) illustrates an exemplary embodiment of insertion system 30 having a lead 34 surrounded by an external insertion tool 32 such that insertion tool 32 does not penetrate skull 31.

In an exemplary embodiment, an exemplary insertion tool can be positioned external with respect to the lead or internal with respect to the lead. FIG. 3(c) illustrates exemplary external insertion tool cross sections illustrating the geometric relationship of the insertion tool with respect to an exemplary internal lead. Cross section views 301, 302, 303, and 304 represent cross section views of an external insertion tool surrounding an exemplary lead such that both the lead and the insertion tool define similar geometries. Thus, view 301 represents a circular geometry of an exemplary internal lead 134 and an exemplary external insertion tool 132; view 302 represents a square geometry of an exemplary internal lead 234 and an exemplary external insertion tool 232; view 303 represents an elliptical geometry of an exemplary internal lead 334 and an exemplary external insertion tool 332; and view 304 represents a triangular geometry of an exemplary internal lead 434 and an exemplary external insertion tool 432.

For improving insertion angle control, an exemplary insertion tool (e.g., a syringe) is used defining a non-rotatably symmetric cross-section geometry. As shown in exemplary cross section views 305, 306 and 307, the geometry defined by a cross section of an exemplary external insertion tool can be different from the geometry defined by a cross section of an exemplary internal lead. Cross section view 305 illustrates an exemplary circular external insertion tool 532 surrounding an exemplary square lead 534. View 306 illustrates an exemplary circular external insertion tool 632 surrounding an exemplary elliptical lead 634. View 307 illustrates an exemplary circular external insertion tool 732 surrounding an exemplary triangular lead 734. Although reference is being made to an internal lead, the previously described geometric embodiments are suitable for alternate embodiments such as an external lead engaged with an insertion tool positioned internal with respect to the lead.

### EXAMPLE 2

In an exemplary embodiment, the present disclosure provides for a brain stimulation system including a pre-curved insertion tool defining a substantial arc of a circle geometry in combination with a relatively soft flexible lead that can be temporarily engaged with the insertion tool during implantation and then subsequently detached. In an exemplary embodiment, the insertion tool is a guide wire. In an exemplary embodiment associated with the present disclosure, a guide wire in combination with a tube-shaped lead having a closed proximal end enables fixation of the guide wire during the placement and/or implantation procedure and facilitates effective detachment of the guide wire once reaching a target location. The insertion tool can be removed at the end of the implantation procedure. In an exemplary embodiment, additional insertion tools can be used during implantation in order to increase mechanical strength of the overall construction and thus improving insertion accuracy.

A particular advantage associated with utilizing a soft lead as described and illustrated in FIGS. 4(a)-4(c) includes but is not limited to the lead having substantially similar mechanical properties as the target brain tissue thus at least avoiding some undesired brain stimulation and/or contact. This may significantly reduce the chances of causing unwanted harm during the insertion and/or stimulation process.

FIG. 4(a) illustrates an exemplary insertion system 40 associated with the present disclosure. An exemplary system 40 includes an internal insertion tool 42 engaged with an external lead 44. Insertion tool 42 can be a guide wire adapted to guide soft lead 44 to a particular target location (e.g., a target brain tissue region). In an exemplary embodiment, guide wire 42 is removably engaged with respect to lead 44 during insertion and can be detached once lead 44 is implanted to conform with a target tissue region. This allows for precise and effective positioning of lead 44 with respect to target tissue region, such as a portion of a brain surrounded by skull 41.

FIG. 4(b) illustrates an exemplary insertion system 40′ associated with the present disclosure. An exemplary system 40′ includes a first internal insertion tool 42′ positioned internal to lead 44′ and is adapted to guide lead 44′ to reach a target location such as a target brain tissue region within skull 41. Typically, first insertion tool 42′ is a guide wire. System 40′ further includes a second insertion tool 43 positioned internal with respect to lead 44′ and surrounding first insertion tool 42′. In an exemplary embodiment, lead 44′ defines a substantially tube geometry having a closed proximal end. Second insertion tool 43 substantially surrounds first insertion tool 42′.

FIG. 4(c) illustrates exemplary cross section views of first and second insertion tools illustrating the geometric relationship between the second insertion tool surrounding the first insertion tool. Cross section views 401, 402, and 403 represent cross section views of a second external insertion tool surrounding an exemplary first insertion tool. Exemplary view 401 illustrates an exemplary first internal insertion tool 142 and an exemplary surrounding second insertion tool 143 such that both insertion tools define a substantially circular geometry. For improving insertion angle control, an exemplary insertion tool (e.g., a syringe) is used defining a non-rotatably symmetric cross section geometry. As shown in exemplary cross section views 402 and 403, the geometry defined by a cross section of an exemplary internal first insertion tool can be different from the geometry defined by a cross section of an exemplary surrounding second internal insertion tool. Cross section view 402 illustrates an exemplary circular second internal insertion tool 243 surrounding an exemplary square first insertion tool 242. View 403 illustrates an exemplary circular second internal insertion tool 343 surrounding an exemplary triangular first insertion tool 342.

### EXAMPLE 3

In an exemplary embodiment, a stimulation system associated with the present disclosure includes a lead fabricated so as to have mechanical characteristics of being stiff but flexible. The lead can be pre-curved having a straight portion and an arc of circle-shaped portion and/or curved. FIG. 5 illustrates an exemplary stimulation system 550 including a lead 554. As illustrated in FIG. 5, system 550 is penetrating a skull 501 associated with an exemplary patient 500 to reach a target tissue region associated with a brain 502. Lead 554 is surrounded by an insertion tool 552. Lead 554 includes a straight portion 555 and a curved portion 556.

The curved portion 556 is adapted to conform with a target tissue region for effective treatment (e.g., stimulation) of the target tissue region while avoiding stimulation and disruption of non-target tissue regions associated with brain 502. In an exemplary embodiment, insertion is performed by a straight syringe-like insertion tool 552. In an exemplary embodiment, syringe 552 straightens the substantially curved portion 556 of lead 554 while lead 554 is positioned internal with respect to syringe 552 during insertion. It further allows lead 554 to follow a curved trajectory after leaving the proximal end of syringe 552.

A particular advantage associated with utilizing an arc of circle and/or helix geometric embodiments as illustrated in FIG. 5 includes restricting the brain tissue damage that may occur to tissue adjacent to the insertion path. The lead includes a lead tip that moves along the insertion path. In the arc of circle and/or helix embodiment, all parts of the lead follow the same path as the lead tip during insertion. An additional advantage includes supporting the traditional standard of substantially linear and/or straight insertion approach to the anatomical target region, except for the final part of the trajectory to obtain the advantage of a more anatomical, orientatable lead for conforming with respect to the geometry of the target tissue region.

In an exemplary embodiment, restricting the brain tissue damage associated with the insertion procedure, as shown in FIG. 5, is accomplished by providing an insertion tool having a proximal end designed in such a way that minimal residual strain is present at the exit opening defined at the proximal end of the insertion tool. In an exemplary embodiment, this is accomplished by aligning the exit channel of a syringe with the desired trajectory of the extending part of the lead (the curved portion). In an exemplary embodiment, the exit channel includes a partially curved portion having the same radius as the pre-curved portion of the lead. Typically, the exit channel length and diameter should be sized and shaped such that the residual strain of the extending part of the lead is minimized. In an exemplary embodiment, the insertion tool is adapted to be removed at the end of the implantation procedure. Similar to the embodiments described with reference to FIGS. 3(c) and 4(c), for improving the control over the insertion angle, an insertion tool having non-rotationally symmetric cross-section (e.g. square, elliptic or triangular) can be employed.

Conforming the lead with the geometry of the target tissue region resulting from a partially curved portion of the lead as shown in FIG. 5 allows for a trajectory that is not longer than necessary for stimulating a target region. Moreover, the difficulty associated with planning of the insertion trajectory is reduced as most of the trajectory is substantially straight. If using a stiff lead, the mechanical properties associated with the (soft) brain tissue are not similarly matched to those of the lead and therefore may lead to increased risk of local brain damage or adverse tissue response during chronic use or insertion.

### EXAMPLE 4

The present disclosure provides for a system including a stiff but flexible pre-curved first insertion tool (e.g. guide wire) having a straight portion and a curved portion in combination with a soft flexible lead that can be temporarily engaged with respect to the guide wire during implantation and then subsequently detached. Similar to the embodiments associated with Example 3 hereinabove, insertion can be performed by an additional straight syringe-like second insertion tool which straightens the curved portion of the guide wire while it is inside the syringe during insertion and allows the guide wire engaged with respect to the lead to follow a curved trajectory after leaving the proximal end of the syringe. In an exemplary embodiment, the first insertion tool (e.g., a guide wire) and the additional second insertion tool (e.g., a syringe or cannula) can be positioned either externally or internally with respect to the lead.

In an exemplary embodiment, restricting the brain tissue damage associated with the insertion procedure, as shown in FIG. 5, is achievable by providing an insertion tool having a proximal end designed in such a way that minimal residual strain is present at the exit opening defined at the proximal end of the insertion tool. In an exemplary embodiment, this is accomplished by aligning the exit channel of the syringe with the desired trajectory of the guide wire (the curved portion). In an exemplary embodiment, the exit channel includes a partially curved portion having the same radius as the pre-curved portion of the guide wire. Typically, the exit channel length and diameter should be sized and shaped such that the residual strain of the extending part of the lead is minimized. In an exemplary embodiment, the insertion tools (e.g., syringe and guide wire) are adapted to be removed at the end of the implantation procedure. Similar to the embodiments described with reference to FIGS. 3(c) and 4(c), for improving the control over the insertion angle, an insertion tool having non-rotationally symmetric cross-section (e.g. square, elliptic or triangular) can be employed.

### EXAMPLE 5

In an exemplary embodiment, a stimulation system associated with the present disclosure includes a lead similar to that as described with respect to Example 1 hereinabove except the lead defines a substantially helical shape (i.e., cork-screw-shape) as shown in FIG. 8. FIG. 8 illustrates an exemplary patient 800 having a skull 801 enclosing a brain 802. A cork-screw-shaped lead 884 penetrates skull 801 to reach and conform with a target tissue region associated with an exemplary brain 802. In an exemplary embodiment, as in Example 1, the lead is stiff and pre-curved. A particular advantage associated with Example 1 and 5 includes improved conforming with the target tissue region associated with the stimulation volume in case of large-diameter physiological targets.

### EXAMPLE 6

In an exemplary embodiment, an insertion system associated with the present disclosure includes a guide wire similar to the guide wire as described with respect to Example 5 hereinabove except the guide wire defines a substantial helical shape (i.e., cork-screw-shape). The guide wire is a stiff pre-curved guide wire and can be utilized in combination with a soft flexible lead that can be temporarily engaged with respect to the guide wire during implantation and then detached.

### EXAMPLE 7

In an exemplary embodiment, a stimulation system associated with the present disclosure includes a lead similar to the lead as described with respect to Example 3 hereinabove except the lead is a stiff pre-curved lead having a straight portion and a helical (i.e., cork-screw-shaped) portion as illustrated in FIG. 9. FIG. 9 illustrates an exemplary patient 900 having a skull 901 enclosing a brain 902. An exemplary lead 994 penetrates skull 901 to reach and conform with a target tissue region associated with brain 902. Lead 994 is engaged temporarily with respect to an insertion tool 992 for guiding lead 994 to the target tissue region. Lead 994 includes a straight (i.e., substantially linear) portion 995 and a helical shaped (i.e., cork-screw-shaped) portion 996.

Similar to the embodiments described with reference to Example 3, insertion of lead 994 can be accomplished using a straight syringe-like insertion tool which straightens the cork-screw-shaped part of the lead while it is inside the syringe during insertion and allows the lead to follow a curved or corkscrew trajectory after leaving the proximal end of the syringe. To achieve restricting of brain tissue damage, the proximal end of the insertion tool should be designed such that minimal residual strain is present in the extending part of the lead. Aligning the exit channel of the syringe with the desired trajectory of the extending part of the lead allows for strain minimization.

With reference to FIG. 6, an exemplary lead 606 includes a straight portion 607 and a helix portion 608. In an exemplary embodiment, helix portion 608 creates a circular curvature defining a diameter of 2R. The curvature is defined such that an extended projection of straight portion 607 substantially aligns with the outer circumference of helix portion of 608. Thus, extended projection of straight portion 607 does not align with the central axis of helix portion 608. A top view illustrating the relationship of 607 along the circumferential edge of 608 is shown with respect to FIG. 6.

### EXAMPLE 8

In an exemplary embodiment, an insertion system associated with the present disclosure includes a lead similar to the lead as described with respect to Example 7 hereinabove except the system includes a stiff pre-curved guide wire having a straight portion and a helical shaped (i.e., cork-screw-shaped) portion, in combination with a soft flexible lead that can be temporarily engaged with respect to the guide wire during implantation and then detached. As previously described in Example 5, the stiff pre-curved guide wire can be utilized in combination with a soft flexible lead that can be temporarily engaged with respect to the guide wire during implantation and then detached.

### EXAMPLE 9

In an exemplary embodiment, an insertion system associated with the present disclosure includes a lead similar to the lead as described with respect to Examples 1, 3, 5 and 7 hereinabove except the lead is a non-pre-curved substantially soft and flexible lead having means for temporarily inducing (in a controlled manner) transversal mechanical strain at least in its proximal portion during insertion and then releasing the strain upon release from the insertion tool. A particular advantage associated with this embodiment includes improved insertion force control while passing the curved lead (or curved portion of the lead) through the straight insertion tool (e.g., a syringe).

### EXAMPLE 10

In an exemplary embodiment, an insertion system associated with the present disclosure includes a lead similar to that as described with respect to Example 9 hereinabove except the transversal mechanical strain is generated by a number of wires running through the lead in a longitudinal direction from the distal end to a proximal end. FIG. 10 illustrates an exemplary flexible lead 1000 associated with the present disclosure, including a plurality of wires 1001 running through the lead in a longitudinal direction from a distal end 1003 to a proximal end 1002. Wires 1001 are adapted to induce transversal mechanical strain.

### EXAMPLE 11

In an exemplary embodiment, a stimulation system associated with the present disclosure includes a guide wire similar to the guide wire as described with respect to Examples 2, 4, 6 and 8 hereinabove except the system includes a non-pre-curved flexible guide wire having means for temporarily inducing (in a controlled manner) transversal mechanical strain at least in its proximal portion during insertion. A particular advantage associated with this embodiment includes improved insertion force control while passing the curved lead (or curved portion of the lead) through the straight insertion tool (i.e., a syringe).

### EXAMPLE 12

In an exemplary embodiment, an insertion system associated with the present disclosure includes a lead similar to the lead as described with respect to Example 11 hereinabove except the transversal mechanical strain is generated similarly to Example 10 by a number of wires running through the guide wire in a longitudinal direction from the distal end to a proximal end.

With reference to FIGS. 6 and 7, particular components of the exemplary embodiments of a system associated with the present disclosure as described with reference to Examples 1-12 are described in further detail. FIG. 6 illustrates an exemplary innermost guide wire 601 having an essentially straight portion 602 distal to an anatomical target or target tissue region, and a curved portion 603 defining a radius of curvature R proximal to the anatomical target. In a further exemplary embodiment, an innermost guide wire can be entirely curved (i.e., arc of a circle curvature) as shown by exemplary curved guide wire 604. An exemplary system utilizing a completely curved guide wire 604 further includes an insertion piece 605 defining a similarly curved inner portion defining a radius R equal to that of the guide wire.

In a further exemplary embodiment, an innermost guide wire 606 is included in an exemplary system associated with the present disclosure. Guide wire 606 includes an essentially straight portion 607 distal to the anatomical target (i.e., target tissue region), and a helix portion 608 defining a helix curvature R and helix pitch h proximal to the anatomical target. For mechanical design and stress distribution motives, the straight portion 607 should be parallel to the helix axis of the helix portion 608 and included in the cylindrical surface that contains the helix portion 608.

Still referring to FIG. 6, in an exemplary embodiment, the insertion system may include an outermost guide tube 609 including a straight tube with axial opening 610. Guide tube 609 is appropriate for guide wires of type 601 or 604. In a further embodiment, outermost guide tube 609 is a straight tube with a lateral opening 611. An embodiment including a tube 609 with an opening 611 is effective for use in cooperation with a helix type of insertion 606. Typically, the inclination of the inner wall of the opening 611 defines an angle alpha as illustrated in FIG. 6. Angle alpha typically is equal to the angle defined by arctan(h/2R), such that h and 2R are associated with the angle of the helix of an exemplary helix portion 608. In an exemplary embodiment, opening 611 is inclined an exit angle alpha equal to the angle curvature of helix portion 608.

In an exemplary embodiment, a lead 612 can optionally consist of a main flexible body 613 and a head 614. Typically, an inner cross-section 615 of body 613 and head 614 is adapted to orient an associated guide wire and/or guide tube relative to an anatomical target (i.e., target tissue region).

FIG. 7 illustrates an exemplary insertion architecture. In an exemplary embodiment, a system associated with the present disclosure includes a positioning apparatus allowing for positioning the insertion system with respect to a skull 704. A positioning apparatus can be essentially identical to existing equipment, including but not limited to guiding tools for stereotactic frames or equivalent tools thereof. An exemplary positioning apparatus is depicted schematically in FIG. 7 as positioning apparatus 703. Apparatus 703 is adapted to allow for insertion of an exemplary lead 612 along an essentially straight trajectory 702 to reach an exemplary anatomical target 706. In an exemplary embodiment, lead 612 is guided to reach and conform with target region 706 along an essentially curved trajectory 705.

In an exemplary embodiment, insertion essentially occurs as follows: an outermost guide tube 609 (second insertion tool) is inserted within a lead 612. An innermost guide wire 601 (first insertion tool), having a tip entering first, is inserted within the outermost guide tube 609 until the tip reaches the opening (610 or 611 as shown in FIG. 6). Guide wire 609 remains entirely inside guide tube 609. Guide tube 609 is actuated until a portion of tube 609 is proximal with respect to the lead as it reaches point 701 positioned on target tissue region 706. Once reaching point 701, a curved trajectory for lead 612 is initiated.

During the curved trajectory inducing portion, guide tube 609 is fixed. The curved trajectory is effectuated by sliding guide wire 601 through guide tube 609 such that the pre-curved shaped portion of guide wire 601 exits from the opening 610/611, thereby initiating a curved portion or a helix along the intended path 705. When the tip of lead 612 has reached the intended position, the lead is maintained in position while the inner guide wire is retracted into the guide tube. The guide tube and guide wire are subsequently retracted.

In an exemplary embodiment, the lead includes at least one electrode. The electrode can be fabricated from a metallic substance or include a metallic coating. A coating must be a continuous, homogenous, heterogeneous or structured material providing at least a benefit of protection at an interface of the lead and the tissue.

Although the present disclosure has been described with reference to exemplary embodiments and implementations thereof, the disclosed systems and methods are not limited to such exemplary embodiments/implementations. Rather, as will be readily apparent to persons skilled in the art from the description provided herein, the disclosed systems and methods are susceptible to modifications, alterations and enhancements without departing from the spirit or scope of the present disclosure. Accordingly, the present disclosure expressly encompasses such modification, alterations and enhancements within the scope hereof.

1. A target tissue insertion system comprising:
(a) a lead adapted to access a target tissue region associated with a target anatomy;
(b) at least a first insertion tool removably engaged with the lead; and
(c) a second insertion tool surrounding the first insertion tool and removeably positioned internally with respect to the lead;
wherein the target tissue region defines a geometry and the lead defines a curved portion adapted to conform with the geometry of the target tissue region, the lead further being substantially tube shaped having an opening at a distal end and a closed portion at a proximal end, wherein the at least first insertion tool and second insertion tool are adapted to be positioned internally with respect to the lead, the lead still further being fabricated to be substantially soft and flexible having mechanical properties similar to properties of the target tissue region and adapted to curve so as to conform with the geometry of the target tissue region after being inserted into the target anatomy;
wherein the first and second insertion tool in combination is adapted to insert the lead into the target anatomy to engage with the target tissue region; and
wherein the first and second insertion tool combination is removable once the lead is positioned with respect to the target tissue region.

2. The system according to claim 1, wherein the insertion tool is adapted to provide guidance and mechanical support to the lead during insertion.

3. The system according to claim 1, wherein the lead accesses the target tissue region to perform a function selected from the group consisting of stimulating the target tissue region, recording activity associated with the target tissue region and delivering a drug and/or chemical to the target tissue region.

4. (canceled)

5. (canceled)

6. The system according to claim 1, wherein the target tissue region is at least a portion of a brain enclosed within a skull of a patient.

7. (canceled)

8. (canceled)

9. (canceled)

10. (canceled)

11. (canceled)

12. (canceled)

13. (canceled)

14. The system according to claim 1, wherein the lead defines a lead tip that moves along a path through the target anatomy during insertion and all parts of the lead follow the same path as the lead tip.

15. (canceled)

16. The system according to claim 1, wherein the at least first insertion tool is a guide wire.

17. (canceled)

18. The system according to claim 1, wherein the first insertion tool is a guide wire and the second insertion tool is a syringe.

19. The system according to claim 1, wherein the first insertion tool is a guide wire and the second insertion tool is a cannula.

20. The system according to claim 1, wherein a cross section of the first insertion tool defines a first geometry and a cross section of the second insertion tool surrounding the first insertion tool defines a second geometry and the first and second geometries are similar.

21. The system according to claim 1, wherein a cross section of the first insertion tool defines a first geometry and a cross section of the second insertion tool surrounding the first insertion tool defines a second geometry and the geometric relationship between the first and second geometries is a non-circularly symmetric relationship.

22. The system according to claim 21, wherein the second geometry is circular and the first geometry is selected from the group consisting of square, elliptical and triangular.

23. (canceled)

24. (canceled)

25. (canceled)

26. (canceled)

27. The system according to claim 1, wherein the at least first insertion tool includes a substantially straight portion at a distal end with respect to the target tissue region and a curved portion at a proximal end with respect to the target tissue region.

28. The system according to claim 27, wherein the lead includes a straight portion and a curved portion such that the curved portion is at a proximal end with respect to the target tissue region and the straight portion is at a distal end with respect to the target tissue region.

29. (canceled)

30. The system according to claim 28, wherein the second insertion tool is fabricated so as to be substantially inflexible defining a substantially straight trajectory.

31. The system according to claim 30, wherein the first insertion tool and the lead are straightened by the inflexible second insertion tool during insertion and then moving along a substantially curved trajectory path as the second insertion tool is being removed.

32. (canceled)

33. The system according to claim 1, wherein the at least first insertion tool is a guide wire and defines a substantially helical or cork-screw geometry.

34. (canceled)

35. The system according to claim 1, wherein the at least first insertion tool is a guide wire and includes a substantially straight portion at a distal end with respect to the target tissue region and a helical or corkscrew portion at a proximal end with respect to the target tissue region.

36. The system according to claim 1, wherein the lead includes a substantially straight portion at a distal end with respect to the target tissue region and a helical or corkscrew portion at a proximal end with respect to the target tissue region.

37. (canceled)

38. The system according to claim 1, wherein the at least first insertion tool further includes a plurality of wires running through the insertion tool in a substantially longitudinal direction for inducing transversal mechanical strain at least in a distal end of the first insertion tool during insertion.

39. (canceled)
